(19) United States
(12) Patent Application Publication
(10) Pub. No.: US 2010/0204567 A1
(43) Pub. Date: Aug. 12, 2010

(54) ULTRASOUND-GUIDED DELIVERY OF A THERAPY DELIVERY DEVICE TO A PHRENIC NERVE

(75) Inventor: Samer Narouze, Akron, OH (US)

Correspondence Address: TAROLLI, SUNDHEIM, COVELL & TUMMINO L.L.P., 1300 EAST NINTH STREET, SUITE 1700, CLEVELAND, OH, 44114, US

(73) Assignee: The Cleveland Clinic Foundation

(21) Appl. No.: 12/702,078

(22) Filed: Feb. 8, 2010

Related U.S. Application Data
(60) Provisional application No. 61/150,808, filed on Feb. 9, 2009.

(51) Int. Cl. A61B 8/13

(52) U.S. Cl. 600/424

(57) ABSTRACT

A method for guiding a therapy delivery device to a phrenic nerve of a subject includes: (a) selecting a target portion of the phrenic nerve; (b) using ultrasound imaging to obtain an ultrasound image of anatomical structures relevant to the target portion; (c) determining an implantation pathway based on the ultrasound image, the implantation pathway defining a trajectory that avoids the relevant anatomical structures and extends between an insertion point on the skin of the subject and the target portion; (d) inserting an introducer into the insertion point, the introducer including a bevel located at a distal end thereof; (e) navigating the introducer through the implantation pathway until the distal tip is positioned adjacent or proximate to the target portion; and (f) advancing the therapy delivery device through the introducer to the target portion of the phrenic nerve. Steps (d)-(f) are performed using real-time ultrasound imaging.

### RELATED APPLICATION

This application claims priority from U.S. Provisional Patent Application Ser. No. 61/150,808 filed Feb. 9, 2009 the subject matter of which is incorporated hereby incorporated by reference in its entirety.

### TECHNICAL FIELD

The present invention relates generally to neuromodulatory methods, and more particularly to an ultrasound-guided approach for modulating the activity of a phrenic nerve.

### BACKGROUND OF THE INVENTION

Surgical procedures are often performed by skilled individuals, such as surgeons. The surgeons can perform various surgical procedures based upon their training and past experience, augmented by study of a particular patient. Nevertheless, various portions of a particular patient may be difficult to examine or identify depending upon the area of the anatomy to be examined and the positioning of the patient.

The anatomical structures surrounding the phrenic nerve, for example, comprise various critical structures in close proximity to the phrenic nerve. The anatomy surrounding the phrenic nerve presents a number of complications potentially associated with access to the phrenic nerve, some of which can be life-threatening.

Surgical techniques used to access and treat the phrenic nerve include the use of computerized tomography (CT) and magnetic resonance imaging (MRI). These techniques are not practical in clinical practice, however, as they are time consuming, cost-ineffective, and involve radiation exposure. Newer approaches, such as fluoroscopy present a reliable technique for identifying bony structures during surgical access to the phrenic nerve. Fluoroscopy cannot, however, identify anatomical structures adjacent to bony structures. Consequently, inadvertent needle placement into the carotid artery, thyroid vessels, trachea, vertebral artery, cervical arteries, nerve roots, thoracic duct, or esophagus can occur when using fluoroscopy to access the phrenic nerve.

### SUMMARY OF THE INVENTION

According to one aspect of the present invention, a method is provided for guiding a therapy delivery device to a phrenic nerve of a subject. One step of the method includes selecting a target portion of the phrenic nerve. A second step of the method includes using ultrasound imaging to obtain an ultrasound image of anatomical structures relevant to the target portion of the phrenic nerve. A third step of the method includes determining an implantation pathway based on the ultrasound image. The implantation pathway defines a trajectory that avoids the relevant anatomical structures and extends between an insertion point on the skin of the subject and the target portion of the phrenic nerve. A fourth step of the method includes inserting an introducer into the insertion point. The introducer includes a bevel located at a distal end thereof. A fifth step of the method includes navigating the introducer through the implantation pathway until the distal tip is positioned adjacent or proximate to the target portion of the phrenic nerve. A sixth step of the method includes advancing the therapy delivery device through the introducer to the target portion of the phrenic nerve. The fourth, fifth, and sixth steps of the method are performed using real-time ultrasound imaging.

According to another aspect of the present invention, a method is provided for treating a medical condition in a subject. One step of the method includes selecting a target portion of the phrenic nerve. A second step of the method includes using ultrasound imaging to obtain an ultrasound image of anatomical structures relevant to the target portion of the phrenic nerve. A third step of the method includes determining an implantation pathway based on the ultrasound image. The implantation pathway defines a trajectory that avoids the relevant anatomical structures and extends between an insertion point on the skin of the subject and the target portion of the phrenic nerve. A fourth step of the method includes inserting an introducer into the insertion point. The introducer includes a bevel located at a distal end thereof. A fifth step of the method includes navigating the introducer through the implantation pathway until the distal tip is positioned adjacent or proximate to the target portion of the phrenic nerve. A sixth step of the method includes advancing the therapy delivery device through the introducer to the target portion of the phrenic nerve. A seventh step of the method includes applying an electrical current to the target portion of the phrenic nerve. The fourth, fifth, and sixth steps of the method are performed using real-time ultrasound imaging.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is a process flow diagram illustrating a method for guiding a therapy delivery device to a phrenic nerve of a subject using real-time ultrasound according to one aspect of the present invention;

FIG. 2 is a perspective view showing an ultrasound transducer positioned near the root of a subject's neck;

FIG. 3 is a perspective view of the subject in FIG. 2 showing an introducer inserted through an implantation pathway;

FIG. 4 is a perspective view of the subject in FIG. 3 showing a therapy delivery device being delivered through the introducer to a target portion of the phrenic nerve; and

FIG. 5 is a process flow diagram illustrating a method for treating a medical condition in subject according to another aspect of the present invention.

### DETAILED DESCRIPTION

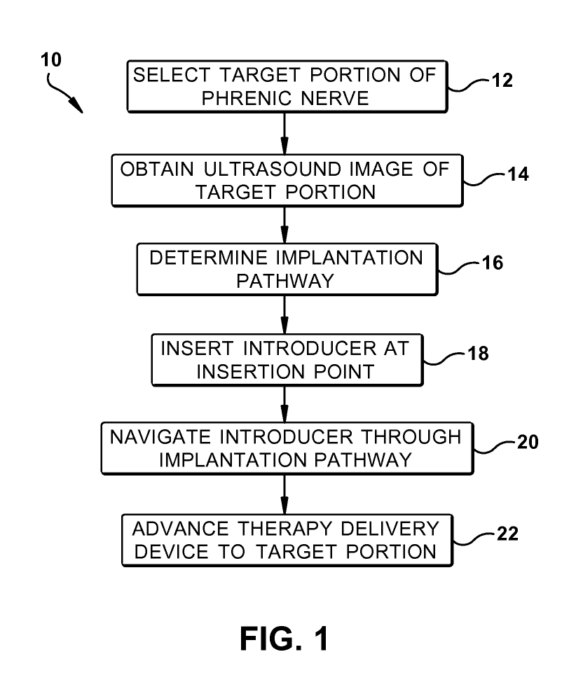

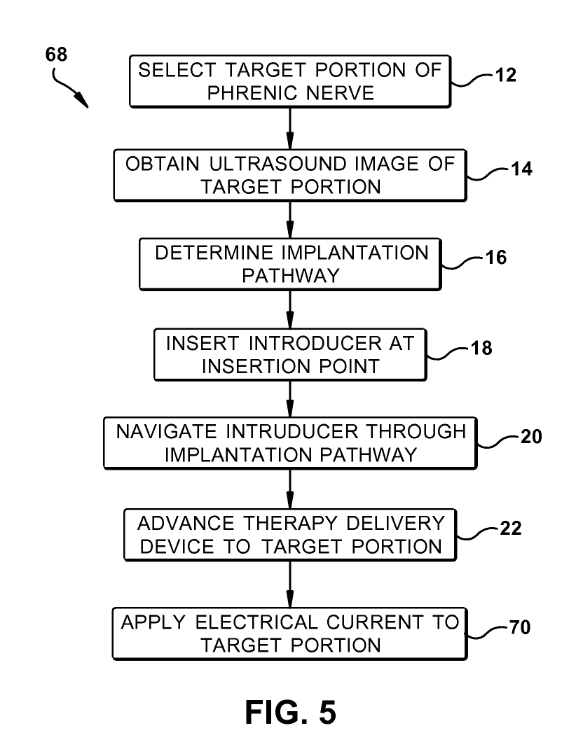

The present invention relates generally to neuromodulatory methods, and more particularly to an ultrasound-guided approach for modulating the activity of a phrenic nerve. As representative of the present invention, FIGS. 1 and 5 illustrate ultrasound-guided methods for delivering a therapy delivery device to a target portion of a phrenic nerve for treatment of a medical condition in a subject. Unlike prior art methods used to guide therapy delivery devices to the phrenic nerve, which require open surgical dissection and/or inadequate imaging techniques, the present invention takes advantages of ultrasound imaging technology to provide a percutaneous approach for guiding a therapy delivery device to a target portion of the phrenic nerve. By using ultrasound technology to guide therapy delivery devices, the present invention provides a surgical technique that substantially reduces the risk of damaging critical anatomical structures during therapy delivery placement at a target portion of the phrenic nerve.

Unless otherwise defined, all technical terms used herein have the same meaning as commonly understood by one of ordinary skill in the art to which the present invention pertains.

In the context of the present invention, the term “medical condition” can refer to both infection- and non-infection-induced diseases and dysfunctions of the respiratory system. Non-limiting examples of medical conditions can include asthma, chronic obstructive pulmonary disease, cystic fibrosis, bronchiolitis, pneumonia, pulmonary thromboembolism, spinal cord injuries, paralysis, bronchitis, emphysema, adult respiratory distress syndrome, allergies, brochiectasis, bronchopulmonary displasia, Chlamydia pneumonia, chronic bronchitis, chronic lower respiratory diseases, croup, familial emphysema, high altitude pulmonary edema, idiopathic pulmonary fibrosis, interstitial lung disease, lymphangioleiomyomatosis, neonatal respiratory distress syndrome, parainfluenza, pleural effusion, pleurisy, pneumothorax, primary pulmonary hypertension, psittacosis, pulmonary edema, pulmonary embolism, pulmonary hypertension, Q fever, respiratory failure, respiratory syncytial virus, sarcoidosis, SARS, smoking, stridor, tuberculosis, acute respiratory distress syndrome, and combinations thereof.

As used herein, the term “phrenic nerve” can refer to the left phrenic nerve, the right phrenic nerve, or both the left and right phrenic nerves.

As used herein, the terms “modulate” or “modulating” can refer to causing a change in neuronal activity, chemistry and/or metabolism. The change can refer to an increase, decrease, or even a change in a pattern of neuronal activity. The terms may refer to either excitatory or inhibitory stimulation, or a combination thereof, and may be at least electrical, biological, magnetic, optical or chemical, or a combination of two or more of these. The terms can also be used to refer to a masking, altering, overriding, or restoring of neuronal activity.

As used herein, the term “subject” can refer to any warm-blooded organism including, but not limited to, human beings, pigs, rats, mice, dogs, goats, sheep, horses, monkeys, apes, rabbits, cattle, etc.

As used herein, the terms “treat” or “treating” shall have their plain and ordinary meaning to one skilled in the art of pharmaceutical or medical sciences. For example, “treat” or “treating” can mean to prevent or reduce chronic hiccups in a subject.

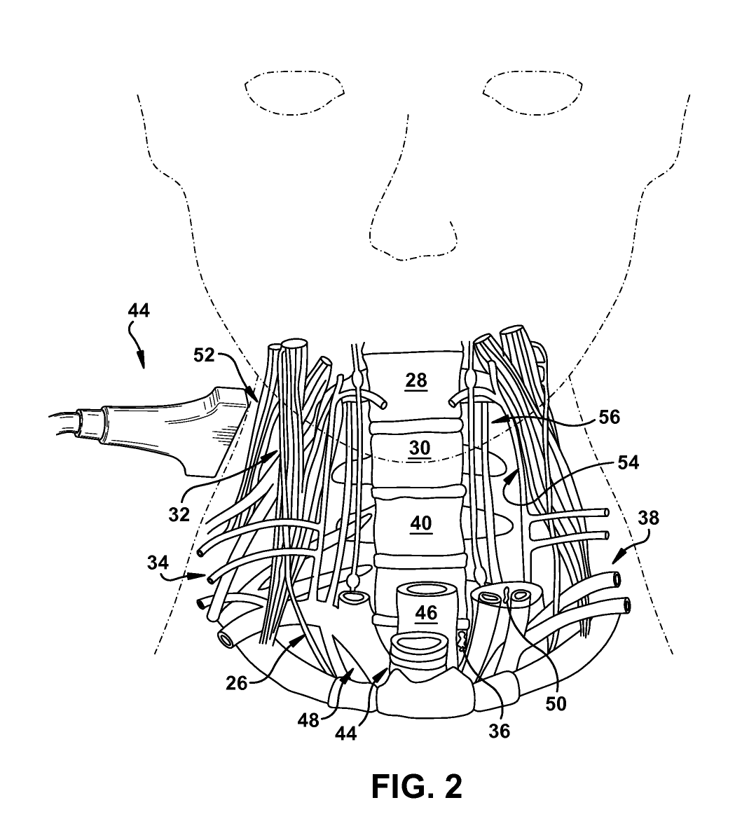

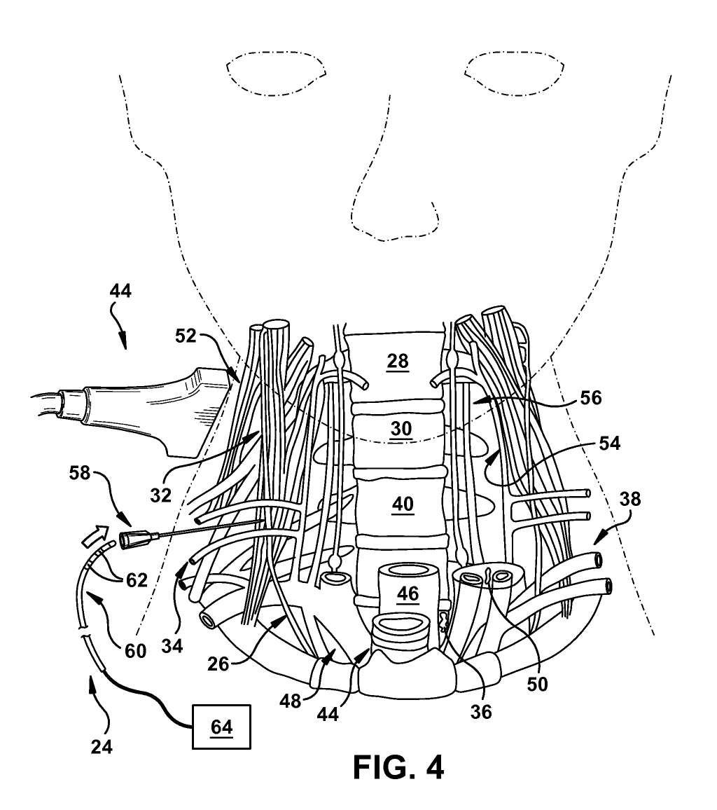

FIG. 1 is a process flow diagram illustrating one aspect of the present invention. In FIG. 1, a method 10 is provided for guiding a therapy delivery device 24 (FIG. 4) to a target portion of the phrenic nerve 26 (FIG. 2) of a subject. The phrenic nerve 26 arises chiefly from the fourth cervical (C4) 28 ventral ramus (not shown), but also the ventral rami of the third (not shown) and fifth cervical (C5) 30 spinal nerves contribute to the formation of the phrenic nerve. The phrenic nerve 26 is formed at the upper part of the lateral border of scalenus anterior muscle 32, and descends almost vertically across its anterior surface behind the prevertebral fascia (not shown). The phrenic nerve 26 descends posterior to sternocleidomastoid (not shown) and the inferior belly of the omohyoid muscles (not shown), as well as the internal jugular vein (not shown), transverse cervical and suprascapular arteries 34, and on the left side thoracic duct 36. At the root of the neck, the phrenic nerve 26 runs anterior to the second part of the subclavian artery 38 from which it is separated by the scalenus anterior muscle 32 (on the left side, the phrenic nerve passes anterior to the first part of the subclavian artery 38). The phrenic nerve 26 descends to the thorax (not shown) and supplies fibrous pericardium (not shown), parietal pleura (mediastinal and central part of the diaphragmatic) (not shown), and the diaphragm (not shown).

At Step 12 of the method 10, a target portion of the phrenic nerve 26 is selected. The target portion can include any section of the phrenic nerve 26, such as a portion of the phrenic nerve at the level of the sixth cervical vertebra (C6) 40. The target portion of the phrenic nerve 26 can be chosen based on a variety of factors, including a particular medical condition or a location relative to surrounding anatomical structures. For example, some target portions may be appropriate targets for a particular medical condition, but situated so that they cannot be readily accessed because of tortuous or precarious anatomical structures surrounding (or en route to) the phrenic nerve 26. Thus, some other target portion of the phrenic nerve 26 that is more readily accessible may have to be selected.

After selecting a target portion of the phrenic nerve 26, an ultrasound image of the anatomical structures relevant to the target portion is obtained using ultrasound imaging at Step 14. Ultrasound apparatus and related methods for obtaining ultrasound images are known in the art and can include linear or curved array transducers capable of producing, two-, three-, or four-dimensional images. One example of such an apparatus is the Sonosite 180 PLUS ultrasound system (not shown) with an 11-mm broadband (4-7 MHz) tightly curved array transducer (SONOSITE, Bothell, Wash.). Before obtaining the ultrasound image, the subject can be positioned in a supine position with his or her neck extended by placing a pillow under the subject's shoulder. Using aseptic technique, an ultrasound transducer 42 (FIG. 2) is then used to obtain a short axis image near the root of the subject's neck to identify relevant anatomical structures. For example, the transducer 42 can be used to obtain a short axis image of the subject's neck at the level of the cricoid cartilage (not shown).

Unlike imaging modalities of the prior art that are used to identify anatomical structures (e.g., fluoroscopy, MRI, CT, etc.), the ultrasound image generated at Step 14 clearly displays important anatomical structures, such as soft tissues, nerves, and vessels. Where the target portion comprises the phrenic nerve 26 at the level of C6 40, for example, the ultrasound image can identify important anatomical structures including, but not limited to, the trachea 44, the esophagus 46, the recurrent laryngeal nerve (not shown), the thyroid gland (not shown), the carotid artery 48, the internal jugular vein, the vagus nerve 50, the scalenus anterior muscle 32, the scalenus medius muscle 52, the inferior thyroid vessels 54, the vertebral vessels 56, and the nerve roots in the interscalene groove (not shown).

At Step 16, an implantation pathway is determined based on the ultrasound image obtained at Step 14. The implantation pathway defines a trajectory that avoids the relevant anatomical structures, and extends between an insertion point on the skin of the subject and the target portion of the phrenic nerve 26. Where the target portion comprises a portion of the phrenic nerve 26 at the level of C6 40, for example, the implantation pathway can be determined by first identifying C6. The level of C6 40 can be identified by the characteristic shape of the transverse process and the position of C6 relative to the vertebral artery 56. After identifying C6 40, a trajectory can be identified that: (1) extends between an insertion point near the root of the subject's neck and C6; and (2) avoids the relevant anatomical structures along the implantation pathway.

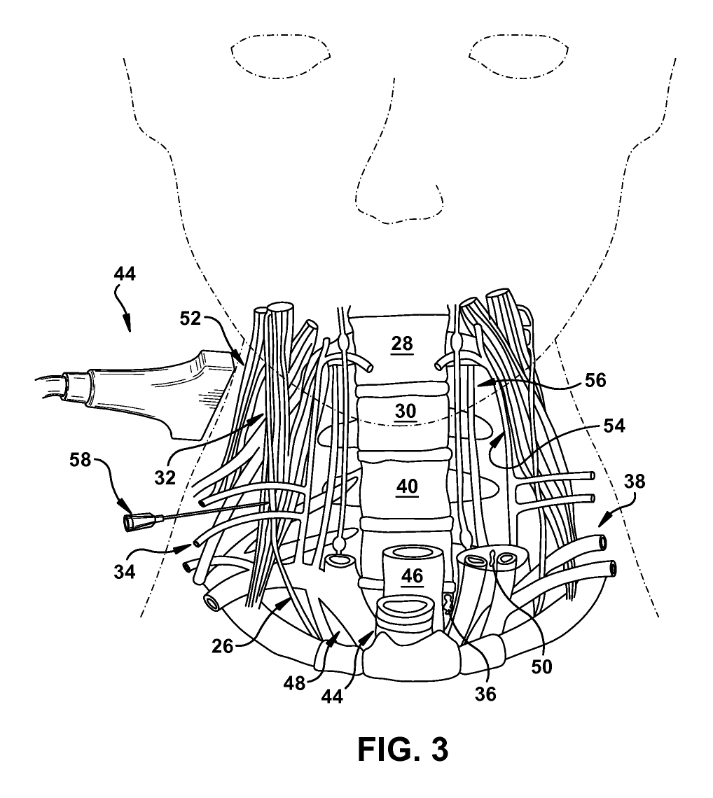

Using real-time ultrasound imaging, an introducer 58 (FIG. 3) is inserted into the insertion point at Step 18. The introducer 58 can comprise any device that is capable of tunneling to, and then delivering, a therapy delivery device 24 (FIG. 5) to a target portion of the phrenic nerve 26. For example, the introducer 58 (FIG. 3) can comprise a needle having a beveled distal tip (not shown in detail). As shown in FIG. 3, the introducer 58 is inserted into the insertion point using an in-plane or out-of-plane approach. Navigation of the introducer 58 through the implantation pathway can be stabilized using a securing mechanism (not shown), such as a guide catheter or adhesive tape. At Step 20, the introducer 58 is navigated through the implantation pathway (e.g., using tactile force) using real-time ultrasound until the distal tip is positioned adjacent or proximate to the target portion. After negative aspiration, placement of the introducer 58 can be checked using real-time fluoroscopy and about 1 ml of contrast agent.

At Step 22, a therapy delivery device 24 (FIG. 4) is delivered to the target portion. The therapy delivery device 24 can include any medical device or apparatus capable of delivering an electrical current to the target portion of the phrenic nerve 26. As shown in FIG. 4, the therapy delivery device 24 can comprise an electrical lead 60 including at least one electrode 62 and being connected to a power source 64. Although the therapy delivery device 24 is shown as being directly connected to the power source 64, it will be appreciated that wireless power sources may also be used to deliver an electric current to the therapy delivery device.

The orientation of the distal tip of the introducer 58 can be adjusted, if needed, depending upon the location of the target portion. For example, the distal tip of the introducer 58 can be rotated so that the bevel is oriented in a cephalad or caudal direction. As the therapy delivery device 24 is advanced through the introducer 58, the ultrasound transducer 42 is moved to obtain a longitudinal image and thereby precisely follow advancement of the therapy delivery device in real-time.

Numerous variations in origin, course, and distribution of the phrenic nerve 26 are known. For example, the phrenic nerve 26 may receive additional roots from one or more of the following nerves: nerve to subclavius (not shown); nerve to sternohyoid (not shown); second or rarely, C6 spinal nerves (not shown); descendens cervicalis (not shown); ansa cervicalis (not shown); and brachial plexus (not shown). The phrenic nerve 26 may receive a branch from cranial nerve (CN) XII (hypoglossal) (not shown), and may communicate with CN XI (spinal accessory) (not shown). Additionally, the phrenic nerve 26 may arise exclusively from the nerve to subclavius. Occasionally, however, the phrenic nerve 26 supplies a branch to the subclavius muscle. Also, in some subjects, the phrenic nerve 26 can be found within the body of the scalene muscles. The consolidation of the phrenic nerve 26 into a single trunk may not occur until it enters the thorax, and the size of the phrenic nerve may vary bilaterally. In view of the phrenic nerve's 26 many anatomical variations, the present invention take advantage of real-time ultrasound to facilitate placement of a therapy delivery device 24 at target portion of the phrenic nerve and thereby minimize the risk of damaging critical anatomical structures when doing so.

FIG. 5 is a process flow diagram illustrating another aspect of the present invention. In FIG. 5, a method 68 is provided for treating a subject with a medical condition. Although the method 68 will be described below in terms of treating a subject with chronic hiccups, it will be appreciated that the method can be used to treat any one or combination of medical conditions described herein.

The steps of the method 68 are substantially identical to Steps 12-22 of the method 10 (FIG. 1) above, except where described below. For example, the method 68 (FIG. 5) can begin by selecting a target portion of the phrenic nerve 26 at Step 12. To treat a subject suffering from chronic hiccups, a target portion of the phrenic nerve 26 at the level of C6 40 can be selected for neuromodulation. After selecting the target portion, an ultrasound image of the anatomical structures relevant to the target portion of the phrenic nerve 26 can be obtained at Step 14. For example, an ultrasound transducer 42 can be used to obtain a short axis image near the root of the subject's neck. The ultrasound image can then be used to identify critical anatomical structures, such as the trachea 44, the esophagus 46, the recurrent laryngeal nerve, the thyroid gland, the carotid artery 48, the internal jugular vein, the vagus nerve 50, the scalenus anterior muscle 32, the scalenus medius muscle 52, the inferior thyroid vessels 54, the vertebral vessels 56, and the nerve roots in the interscalene groove.

At Step 16 of the method 68, an implantation pathway can be determined based on the ultrasound image. Where the target portion comprises the phrenic nerve 26 at the level of C6 40, the implantation pathway can be determined by first identifying C6. The level of C6 40 can be identified by the characteristic shape of the transverse process and the position of C6 relative to the vertebral artery 56. After identifying C6 40, a trajectory can be identified that: (1) extends between an insertion point at the root of the subject's neck and C6; and (2) avoids the relevant anatomical structures along the implantation pathway.

Using real-time ultrasound, an introducer 58 can be inserted into the insertion point at Step 18. The introducer 58 can comprise a needle having a beveled distal tip, for example. The introducer 58 can be inserted at the insertion point using an in-plane or out-of-plane approach to target the phrenic nerve 26 at the level of C6 40. Navigation of the introducer 58 through the implantation pathway can be stabilized using a securing mechanism (not shown), such as a guide catheter or adhesive tape. At Step 20, the introducer 58 can be navigated through the implantation pathway (e.g., using tactile force) using real-time ultrasound until the distal tip is positioned adjacent or proximate to the target portion of the phrenic nerve 26. After negative aspiration, placement of the introducer 58 can be checked using real-time fluoroscopy and about 1 ml of contrast agent.

At Step 22, a therapy delivery device 24 can be delivered to the target portion (FIG. 4). As shown in FIG. 4, the therapy delivery device 24 can comprise an electrical lead 60 including at least one electrode 62 and being connected to a power source 64. With the distal tip of the introducer 58 positioned adjacent or proximate to the target portion of the phrenic nerve 26, the electrical lead 60 can then be advanced through the introducer (indicated by arrow) under real-time ultrasound until at least one electrode 62 of the electrical lead is positioned adjacent or proximate to the target portion.

At Step 70, the power source 64 can be activated so that an electrical current is passed through the electrical lead 60 and into the target portion of the phrenic nerve 26. The electrical current may be episodic, continuous, phasic, in clusters, intermittent, upon demand by the subject or medical personnel, or pre-programmed to respond to a sensor (not shown) (e.g., a closed-loop system). The electrical current can be operated at a constant voltage (e.g., at about 0.1 v to about 25 v), at a constant current (e.g., at about 25 microampes to about 50 milliamps), at a constant frequency (e.g., at about 5 Hz to about 10,000 Hz), and at a constant pulse-width (e.g., at about 50 μsec to about 10,000 μsec). Application of the electrical current can be monopolar, bipolar, or multipolar, depending upon the polarity of the electrical lead 60. The waveform may be, for example, biphasic, square wave, sine wave, or other electrically safe and feasible combinations. Additionally, the electrical current may be applied to the target portion of the phrenic nerve 26 either simultaneously or sequentially.

Delivery of electrical current to the target portion can suppress the occurrence of hiccups experienced by the subject by “blocking” nerve impulse transmission through the phrenic nerve 26. As unregulated and increased nerve transmission through the phrenic nerve 26 to the diaphragm is essential for the body to propagate hiccups, blocking nerve impulse transmissions through the phrenic can prevent or diminish the occurrence of hiccups experienced by the subject. Upon delivery of the electrical current to the electrical lead 60, the position of the electrical lead, or frequency of electrical current being delivered to the electrical lead, may then be adjusted until the subject reports (or is observed) experiencing fewer hiccups. Once satisfactory, the electrical lead 60 can be self-anchored or anchored to the deep tissues and then tunneled to an infraclavicular area (not shown) where an IPG (not shown) can be implanted.

From the above description of the invention, those skilled in the art will perceive improvements, changes and modifications. For example, it will be appreciated that wireless technologies (e.g., RF devices) can be used to deliver power to the therapy delivery device 24. Such improvements, changes, and modifications are within the skill of the art and are intended to be covered by the appended claims.

Having described the invention, I claim:

1. A method for guiding a therapy delivery device to a phrenic nerve of a subject, said method comprising the steps of:
(a) selecting a target portion of the phrenic nerve;
(b) using ultrasound imaging to obtain an ultrasound image of anatomical structures relevant to the target portion of the phrenic nerve;
(c) determining an implantation pathway based on the ultrasound image, the implantation pathway defining a trajectory that avoids the relevant anatomical structures and extends between an insertion point on the skin of the subject and the target portion of the phrenic nerve;
(d) inserting an introducer into the insertion point, the introducer including a bevel located at a distal end thereof;
(e) navigating the introducer through the implantation pathway until the distal tip is positioned adjacent or proximate to the target portion of the phrenic nerve; and
(f) advancing the therapy delivery device through the introducer to the target portion of the phrenic nerve;
wherein steps (d)-(f) are performed using real-time ultrasound imaging.

2. The method of claim 1, wherein the target portion of the phrenic nerve is located between the cervical plexus and the level of C7.

3. The method of claim 2, wherein the target portion of the phrenic nerve is located at the level of C6.

4. The method of claim 1, wherein said step of using ultrasound imaging to obtain an ultrasound image further comprises the steps of:
obtaining a short axis ultrasound image of the subject's neck at the cricoid cartilage level; and
identifying a plurality of anatomical structures selected from the group consisting of the trachea, the esophagus, the recurrent laryngeal nerve, the thyroid gland, the carotid artery, the internal jugular vein, the vagus nerve, the scalenus anterior muscle, the scalenus medius muscle, and the interscalene groove.

5. The method of claim 1, wherein said step of navigating the introducer through the implantation pathway further comprises providing a securing mechanism to stabilize the introducer during navigation through the implantation pathway.

6. The method of claim 1, wherein said step of inserting an introducer into the insertion point is performed using an in-plane or out-of-plane approach.

7. The method of claim 1, wherein said step of advancing the therapy delivery device through the introducer further comprises the steps of:
directing the bevel of the introducer caudally; and
obtaining a longitudinal image of the scalenus muscle area to follow the therapy delivery device during advancement to the target portion of the phrenic nerve.

8. A method for treating a medical condition in a subject, said method comprising the steps of:
(a) selecting a target portion of the phrenic nerve;
(b) using ultrasound imaging to obtain an ultrasound image of anatomical structures relevant to the target portion of the phrenic nerve;
(c) determining an implantation pathway based on the ultrasound image, the implantation pathway defining a trajectory that avoids the relevant anatomical structures and extends between an insertion point on the skin of the subject and the target portion of the phrenic nerve;
(d) inserting an introducer into the insertion point, the introducer including a bevel located at a distal end thereof;
(e) navigating the introducer through the implantation pathway until the distal tip is positioned adjacent or proximate to the target portion of the phrenic nerve;
(f) advancing the therapy delivery device through the introducer to the target portion of the phrenic nerve; and
(g) applying an electrical current to the target portion of the phrenic nerve;
wherein steps (d)-(f) are performed using real-time ultrasound imaging.

9. The method of claim 8, wherein said step of applying an electrical current to the target portion of the phrenic nerve further comprises delivering an amount of electrical current sufficient to prevent or mitigate an acute medical condition.

10. The method of claim 8, wherein the target portion of the phrenic nerve is located between the cervical plexus and the level of C7.

11. The method of claim 10, wherein the target portion of the phrenic nerve is located at the level of C6.

12. The method of claim 8, wherein said step of using ultrasound imaging to obtain an ultrasound image further comprises the steps of:
obtaining a short axis ultrasound image of the subject's neck at the cricoid cartilage level; and
identifying a plurality of anatomical structures selected from the group consisting of the trachea, the esophagus, the recurrent laryngeal nerve, the thyroid gland, the carotid artery, the internal jugular vein, the vagus nerve, the scalenus anterior muscle, the scalenus medius muscle, and the interscalene groove.

13. The method of claim 8, wherein said step of navigating the introducer through the implantation pathway further comprises providing a securing mechanism to stabilize the introducer during navigation through the implantation pathway.

14. The method of claim 8, wherein said step of inserting an introducer into the insertion point is performed using an in-plane or out-of-plane approach.

15. The method of claim 8, wherein said step of advancing the therapy delivery device through the introducer further comprises the steps of:
directing the bevel of the introducer caudally; and
obtaining a longitudinal image of the scalenus muscle area to follow the therapy delivery device during advancement to the target portion of the phrenic nerve.
